(19) United States
(12) Patent Application Publication
(10) Pub. No.: US 2026/0049354 A1
(43) Pub. Date: Feb. 19, 2026

(54) NANOPORE-BASED MULTIPLEXED REPORTER ASSAY SYSTEM AND METHOD

(71) Applicant: ATTAGENE, INC., RESEARCH TRIANGLE PARK, NC (US)

(72) Inventors: SERGEI S. MAKAROV, MORRISVILLE, NC (US); ALEXANDER VLADIMIROVICH MEDVEDEV, DURHAM, NC (US)

(21) Appl. No.: 19/104,411

(22) PCT Filed: Aug. 18, 2023
(86) PCT No.: PCT/US2023/072526

§ 371 Date: Feb. 18, 2025

Related U.S. Application Data
(60) Provisional application No. 63/371,808, filed on Aug. 18, 2022.

(51) Int. Cl. C12Q 1/6869; C12Q 1/6806; C12Q 1/6897

(52) U.S. Cl. CPC C12Q 1/6869; C12Q 1/6806; C12Q 1/6897

(57) ABSTRACT

A method for parallel (multiplexed) assessment of multiple reporter constructs in living cells is described, in which a population of RTU constructs is introduced into assay cells, cellular RNA is isolated and annealed to a detection oligonucleotide that contains a sequence complementary to the RTU reporter sequence and an adapter sequence conducive for RNA sequencing by a nanopore sequencing device, optionally with amplification of reporter RNA transcripts before detection nucleotide annealing, sequencing annealed reporter RNA or its amplified cDNA using a nanopore sequencing instrument, analyzing the sequencing data to identify and tally processing tags of individual RTUs, and calculating and generating an RTU activity profile as the frequency of counted processing tags. Methods are also described for real-time controlling of quality of multiplexed reporter construct detection, and for assessment of multiple reporter constructs and gene expression in living cells.

### CROSS-REFERENCE TO RELATED APPLICATION

The benefit under 35 USC § 119 of U.S. Provisional Patent Application 63/371,808 filed Aug. 18, 2022 in the name of Sergei S. Makarov for “DYNAMIC REAL-TIME TRANSCRIPTION FACTOR ACTIVITY ASSAY SYSTEM AND METHOD” is hereby claimed. The disclosure of U.S. Provisional Patent Application 63/371,808 is hereby incorporated herein by reference, in its entirety, for all purposes.

### BACKGROUND

### Field of the Invention

The present disclosure relates to systems, kits, and methods for the simultaneous assessment of multiple reporter constructs in living cells.

### Description of the Related Art

To accommodate changes in environments in which they reside, cells adjust their gene expression. Such adaptive regulation is according by the cellular network of signaling pathways transmitting regulatory signals from the cell's periphery to genes. Malfunctions and aberrations of the cellular signaling can cause pathological changes in gene expression and disease.

With the advent of systems biology, researchers can now evaluate gene expression on a global scale. However, since the direct manipulation of genes is challenging, the contemporary paradigm of drug development now emphasizes targeting signaling pathways governing gene expression. In this effort, the primary challenge is to accurately identify disease-associated pathways, which necessitates tools with high-content capabilities to dissect the cellular signaling network.

Historically, reporter gene assays have been the gold standard for analyzing cellular signaling. These assays utilize reporter gene constructs comprising a readily assayable reporter gene (such as luciferase or green fluorescent protein) under the control of a pathway-responsive promoter. When cells are transfected with these reporter constructs, they express reporter proteins proportionate to the pathway's activity. However, due to a limited range of reporter proteins, these assays allow assessing only a few (typically just one) signaling pathway at a time.

Romanov et al. (U.S. Pat. No. 7,700,284; S. Romanov, et al., “Homogeneous reporter system enables quantitative functional assessment of multiple transcription factors”, Nature Methods 5, 253-260 (2008)) sought to circumvent this bottleneck by introducing a multiplexed reporter system, known as FACTORIAL®, composed of a set of reporter constructs termed reporter transcription units (RTU). Similar to conventional reporter gene constructs, RTUs harbor a reporter sequence regulated by a pathway-sensitive promoter. But, uniquely, RTU activity is gauged via their RNA transcripts.

To enable a uniform multiplexed detection, all RTUs use an identical reporter sequence, distinguished by a short ‘processing tag’. In the preferred embodiment, such tag represents a unique restriction enzyme site located variably within the reporter sequences. To detect RTU transcripts, cellular RNA is amplified by reverse transcription followed by PCR with a single pair of RTU-specific primers. Subsequently, the cDNAs are cleaved by the restriction enzyme, and the resultant DNA fragments are separated by electrophoresis, yielding an electropherogram profile indicative of RTU activity.

An alternative strategy for multiplexed detection of cis-regulatory sequences, like gene promoters and enhancers, is a massive parallel reporter assay (MPRA) (S. R. Grossman, et al., Systematic dissection of genomic features determining transcription factor binding and enhancer function. Proc. Natl. Acad. Sci. U.S.A. 114, E1291-E1300 (2017); J. C. Klein, et al., A systematic evaluation of the design and context dependencies of massively parallel reporter assays. Nature Methods 17, 1083-1091 (2020)). Here, the cis-regulatory elements under evaluation are fused to barcoded reporter sequences and transfected into cells. The reporter RNAs are amplified by RT-PCR and the resulting cDNAs are ligated as concatamers into extended DNA strands. These are then sequenced using the Illumina sequencer, with barcode frequency providing insights into the activity of the cis-regulatory elements under assessment.

The art continues to seek improvements in the above-discussed technology.

### SUMMARY

The present disclosure relates to systems, kits, and methods for the simultaneous assessment of multiple reporter constructs in living cells.

The disclosure relates in one aspect to a method for parallel (multiplexed) assessment of multiple reporter constructs in living cells is described, in which a population of RTU constructs is introduced into assay cells, cellular RNA is isolated and annealed to a detection oligonucleotide that contains a sequence complementary to the RTU reporter sequence and an adapter sequence conducive for RNA sequencing by a nanopore sequencing device, optionally with amplification of reporter RNA transcripts before detection nucleotide annealing, sequencing annealed reporter RNA or its amplified cDNA using a nanopore sequencing instrument, analyzing the sequencing data to identify and tally processing tags of individual RTUs, and calculating and generating an RTU activity profile as the frequency of counted processing tags.

In another aspect, the disclosure relates to a method for the parallel assessment of the activity of multiple reporter constructs in living cells, such method comprising:
(a) use of a uniform population of reporter transcription units (RTU), each containing an identical reporter sequence;
(b) distinction of each RTU reporter transcript by a processing tag embedded within the reporter sequence, said processing tag either representing a restriction enzyme site variably located within reporter sequences or a barcoding nucleotide sequence, wherein said tags do not interfere with RTU transcription;
(c) introducing the RTU population into assay cells;
(d) isolating cellular RNA, and annealing same to an RTU detection oligonucleotide, which possesses a sequence complementary to the RTU reporter sequence and a facilitating adapter sequence for RNA sequencing by a nanopore sequencing device;
(e) optionally amplifying reporter RNA transcripts before the annealing (d) of said detection oligonucleotide via consecutive reverse transcription and PCR using RTU-specific primers;
(f) sequencing the annealed reporter RNA or its amplified cDNA through a nanopore sequencing device;
(g) analyzing sequencing output to identify and enumerate processing tags associated with individual RTUs; and
(h) calculating and generating an RTU activity profile as the frequency of the counted processing tags.

In a further aspect, the disclosure relates to a method for real-time controlling of quality of multiplexed reporter construct detection in the above-described method for the parallel assessment of the activity of multiple reporter constructs in living cells, wherein the real-time controlling method comprises:
(a) assessing RTU activity profiles calculated and generated in step (h) at two consecutive time points;
(b) calculating similarity of said RTU activity profiles using a Pearson correlation coefficient or Euclidean distance technique; and
(c) stopping the sequencing once the RTU profiles'similarity reaches a predetermined quality threshold.

Another aspect of the disclosure relates to a method for combined assessment of multiple reporter constructs and gene expression in living cells, such method comprising:
(a) use of a uniform population of reporter transcription units (RTU) and introduction of such RTU population into assay cells according to steps (a)-(c) defined in the above-described method for the parallel assessment of the activity of multiple reporter constructs in living cells;
(b) isolating cellular mRNA;
(c) annealing one part of such isolated mRNA to an RTU detection oligonucleotide according to steps (d)-(e) defined in the above-described method for the parallel assessment of the activity of multiple reporter constructs in living cells;
(d) annealing the other part of the isolated mRNA to an RNA detection oligonucleotide containing a poly(T) sequence complementary to the 3′ poly(A) RNA tail and an adapter sequence facilitating RNA sequencing by a nanopore sequencing device;
(e) mixing the annealed RNAs and sequencing the mix using a nanopore sequencing device, or
(f) alternatively, sequencing the annealed RNAs separately;
(g) analyzing the sequencing output to identify and enumerate processing tags associated with individual RTUs and calculating and generating an RTU activity profile as the frequency of the counted processing tags; and
(h) analyzing the sequencing output to calculate and generate an output of the expression of endogenous mRNA.

The disclosure relates in a further aspect to a kit for nanopore-based assessment of multiple reporter constructs, such kit comprising RTU constructs, detection reagents, and software for analyzing the sequencing output and calculating RTU activity profiles and gene expression according to the various methods of the disclosure.

Other aspects, features and embodiments of the disclosure will be more fully apparent from the ensuing description and appended claims.

### BRIEF DESCRIPTION OF THE DRAWINGS

FIG. 1 schematically depicts a generalized nanopore sequencing process.

FIG. 2 schematically depicts a method for parallel assessment of multiple reporter constructs in living cells, in accordance with one aspect of the present disclosure.

FIG. 3 shows the fidelity of the nanopore-based RTU detection method of the present disclosure as reliably generating data replicating that obtainable using the capillary electrophoresis (CE)-based FACTORIAL® technology of Romanov et al.

FIG. 4 schematically depicts the operation sequence for real time control of the quality of the RTU detection in the methods of the present disclosure.

FIG. 5 schematically depicts the capability of the methods of the present disclosure to combine high-content assessment of cellular signaling with transcriptomics analysis.

### DETAILED DESCRIPTION

The present disclosure relates in various aspects to a novel method and system that empowers the quantitative, multiplexed analysis of reporter constructs via a nanopore sequencing device. This technology expands the capabilities of existing multiplexed reporter techniques and provides a portable technology that is uniquely suited for use by individual researchers.

In various aspects, the disclosure relates to a method for parallel assessment of the activity of multiple reporter constructs in living cells (hereinafter referred to as NanoFACTORIAL). The method of the present disclosure employs a population of reporter constructs termed reporter transcription units (RTU), analogous to the reporter constructs and RTUs that were utilized in the previously discussed technology of Romanov et al.

Within the ambit of the present disclosure, an RTU is defined as a DNA construct incorporating a cis-regulatory element that is functionally linked to a transcribed reporter sequence. This cis-regulatory element can comprise components such as a binding site for a transcription factor (TF), elements regulating RNA stability, gene promoters, gene enhancers, gene silencers, or other DNA regions involved in gene expression regulation.

The RTU population has a uniform design, wherein all RTU constructs employ identical reporter sequences. This uniformity ensures that reporter RNAs are equally susceptible to different experimental settings, e.g., RNA degradation or RT-PCR amplification, thereby ensuring reproducibility of RTU activity profiles in a broad range of experimental conditions.

The RTU reporter transcripts are identifiable through a short nucleotide sequence, termed the ‘processing tag’, located within its reporter sequence. This tag could represent a restriction enzyme site set at varying positions in respective reporter sequences or could be a barcoding nucleotide sequence. It is imperative that the processing tag be of a character that does not impede the RTU transcription.

The unique feature distinguishing the present technology from other multiplexed reporter technologies, such as those of Romanov et al. and MPRA, is a novel RTU detection approach, which entails sequencing the reporter RNA by a nanopore-based sequencing device.

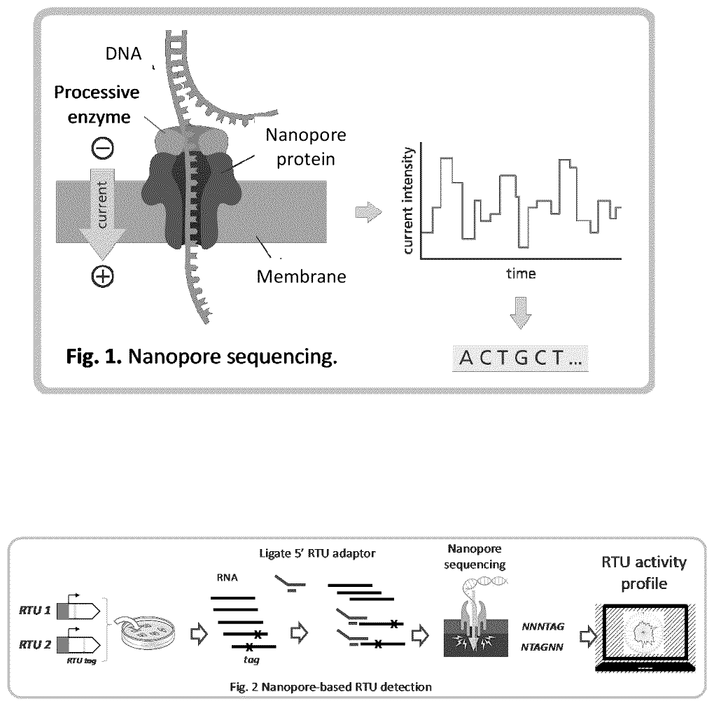

At the core of a nanopore-based sequencing device is a flow cell with membrane-embedded nanopore-forming proteins, such as is for example described in M. Jain, H. E. Olsen, B. Paten, M. Akeson, The Oxford Nanopore MinION: delivery of nanopore sequencing to the genomics community. Genome Biol. 17 (2016). FIG. 1 hereof schematically depicts the generalized nanopore sequencing process. For the sequencing process, the nanopore sequencing RNA or DNA substrates are ligated to sequencing adapters that facilitate the loading of a processive enzyme, which ensures single-nucleotide displacement along the strand. As the sequenced molecule passes through the pore, a sensor detects changes in ionic current caused by the shifting nucleotide sequence within the pore. Such current changes are interpreted computationally as the nucleotide sequence.

Importantly, the nanosequencing approach enables real-time basecalling and immediate access to result (M. Loose, S. Malla, M. Stout, Real-time selective sequencing using nanopore technology. Nat. Methods 13, 751-754 (2016)). Furthermore, sequencing can be stopped once sufficient data is obtained, following which the flow cell can be washed and reused.

The nanopore-based detection of RTU transcripts according to the present disclosure has multiple advantages. First, the nanopore sequencing allows combining the high-content analysis of cellular signaling with transcriptomics assessment of gene expression, a unique feature unattainable with any other technology. Second, the nanopore-based approach is a digital technology that permits controlling the quality of detection output in real time. Third, it replaces an expensive stationary centerpiece detection equipment (such as capillary DNA fragment analyzer or an Illumina sequencer) with a low-cost pocket-size nanopore sequencing device. Further advantages will be more fully apparent from the ensuing disclosure and description.

The method for parallel assessment of multiple reporter constructs in living cells, in accordance with the present disclosure, is schematically depicted in FIG. 2 hereof, and involves the following steps:
Step 1.1. Introducing a population of RTU constructs into assay cells, e.g., via transient or stable transfection, electroporation, or microinjection.
Step 1.2. Isolating cellular RNA and annealing it to a detection oligonucleotide that contains a sequence complementary to the RTU reporter sequence and an adapter sequence conducive for RNA sequencing by a nanopore sequencing device.
Step 1.3. Optionally, amplifying the reporter RNA transcripts before the detection nucleotide's annealing, through sequential reverse transcription and PCR using RTU-specific primers.
Step 1.4. Sequencing the annealed reporter RNA or its amplified cDNA using a nanopore sequencing instrument, such as those commercially available under the trademarks MinION, PromethION, or GridION from Oxford Nanopore Technologies, Oxford, United Kingdom.
Step 1.5. Analyzing the sequencing data to identify and tally processing tags of individual RTUs.
Step 1.6. Calculating and generating an RTU activity profile as the frequency of counted processing tags.

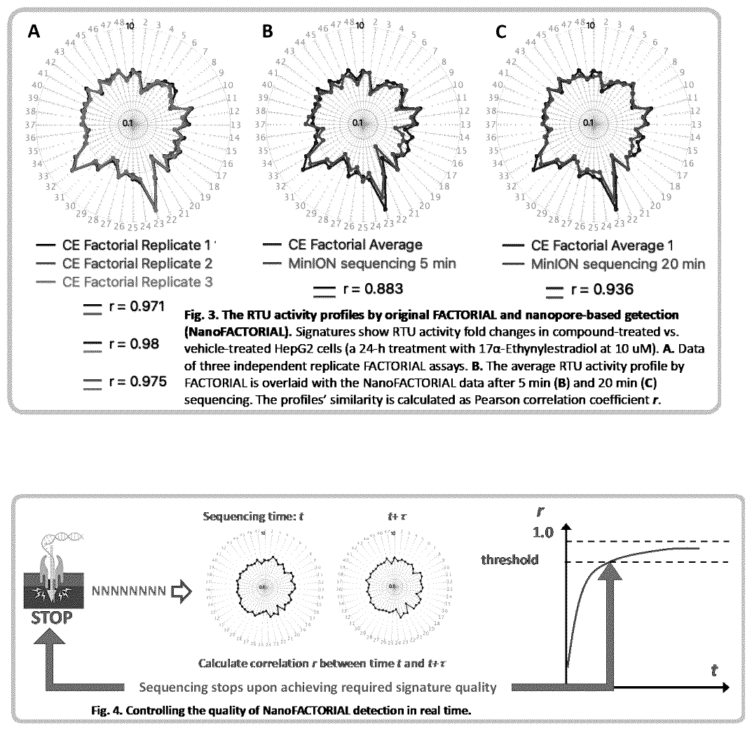

Importantly, the nanopore-based RTU detection method of the present disclosure reliably generates data replicating that obtainable using the original capillary electrophoresis (CE)-based FACTORIAL® technology of Romanov et al., as is illustratively shown in FIG. 3 hereof.

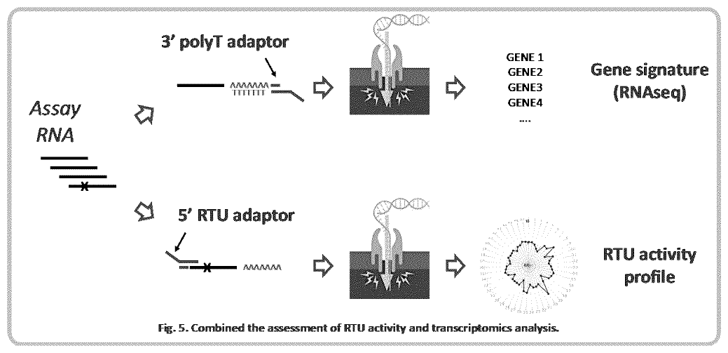

The present disclosure further provides a method for controlling the quality of multiplexed detection. The detection quality in nanopore sequencing is determined by the statistical fluctuations in the count of RTU tags. As the sequencing time increases, these fluctuations decrease, leading to improved detection quality. The output from nanopore sequencing can be basecalled and analyzed in real-time, enabling the real-time monitoring of the RTU activity profile. The RTU activity profiles at consecutive time points can be compared quantitatively, for instance, using the Pearson correlation or Euclidean distance techniques (A. Medvedev, et al., Evaluating biological activity of compounds by transcription factor activity profiling. Sci. Adv. 4, eaar4666 (2018)). As sequencing time increases, the correlation will asymptotically approach the value of 1.0. Consequently, users can set a desired detection quality threshold. Once this threshold is met, sequencing can be stopped, as schematically depicted in FIG. 4 hereof.

Accordingly, the present disclosure contemplates a method for controlling the quality of multiplexed detection, comprising the steps of:
2.1. Analyzing the nanopore sequencing output as outlined in Step 1.6 to calculate and generate the RTU activity profiles at sequential time points.
2.2. Calculating the similarity of RTU activity profiles at these time points using a correlation analysis.
2.3. Terminating sequencing after the correlation coefficient has reached a predetermined value.

A distinctive feature of the methodology of the present disclosure is its ability to combine high-content assessment of cellular signaling with transcriptomics analysis, as is schematically depicted in FIG. 5 hereof. This methodology integrates direct mRNA sequencing using nanopore-based devices, such as nanopore-based devices described, for example, in M. Loose, S. Malla, M. Stout, Real-time selective sequencing using nanopore technology. Nature Methods 13, 751-754 (2016) and M. Seki, et al., Evaluation and application of RNA-Seq by MinION. DNA Res. 26, 55-65 (2019).

The method of the present disclosure in further embodiments may correspondingly comprise the following steps:
Step 3.1. Introducing an RTU population into assay cells, e.g., by methods such as transient or stable transfection, electroporation, or microinjection.
Step 3.2. Isolating cellular mRNA and dividing it into two fractions.
Step 3.3. Annealing one portion of the isolated RNA (fraction A) to an RTU detection oligonucleotide. As specified in Step 1.2, such oligonucleotide should have a sequence complementary to the RTU reporter sequence and an adapter sequence facilitating RNA sequencing by a nanopore sequencing device.
Step 3.4. Optionally, amplifying reporter RNA transcripts before annealing to the detection oligonucleotide, as specified in Step 1.3. Such amplification can be achieved, for example, through sequential reverse transcription and PCR using RTU-specific primers.
Step 3.5. Annealing the other part of the isolated RNA (fraction B) to an RNA detection oligonucleotide containing a poly(T) sequence complementary to the 3′ poly(A) RNA tail, as well as an adapter sequence facilitating RNA sequencing by a nanopore sequencing device.
Step 3.6. Combining the annealed fractions A and B, then sequencing them using a nanopore sequencing device, e.g., a nanopore sequencing device such as those commercially available under the trademarks MinION, PromethION, or GridION from Oxford Nanopore Technologies, Oxford, United Kingdom.
Step 3.7. Alternatively, separately sequencing the annealed fractions A and B.
Step 3.8. Analyzing the sequencing output to identify and count processing tags of individual RTUs and calculating and generating an RTU activity profile.
Step 3.9. Analyzing the sequencing output to calculate and generate an output of expression of endogenous mRNA.

A further aspect of the disclosure relates to a kit for nanopore-based assessment of multiple reporter constructs, such kit comprising RTU constructs, detection reagents, and computer software for analyzing the sequencing output and calculating RTU activity profiles and gene expression according to the methods of the present disclosure, as variously described herein.

The computer software may be provided as a computer program product comprising a non-transitory computer readable storage medium having program instructions embodied therewith, the program instructions executable by a computing device to cause the computing device to carry out computer-implemented operations of analysis, calculation, and generation of corresponding outputs to graphical user interfaces of such computing devices. The displayed outputs to graphical user interfaces of computing devices may include RTU activity profiles, computed measures of similitude of successive or otherwise different RTU activity profiles, outputs of expression of endogenous mRNA, and outputs for stopping sequencing operation of the sequencing device once similarity of RTU profiles reaches a predetermined quality threshold.

Accordingly, the disclosure relates in additional aspects to systems for parallel (multiplexed) assessment of multiple reporter constructs in living cells comprising a nanopore sequencing device operatively coupled to a computing device including a central processing unit (CPU) a computer readable medium in which program instructions are present for carrying out computer-implementable steps in the various methods of the present disclosure.

While the disclosure has been set out herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the invention as hereinafter claimed is intended to be broadly construed and interpreted, as including all such variations, modifications and alternative embodiments, within its spirit and scope.

1. A method for the parallel assessment of the activity of multiple reporter constructs in living cells, said method comprising:
(a) use of a uniform population of reporter transcription units (RTU), each containing an identical reporter sequence;
(b) distinction of each RTU reporter transcript by a processing tag embedded within the reporter sequence, said processing tag either representing a restriction enzyme site variably located within reporter sequences or a barcoding nucleotide sequence, wherein said tags do not interfere with RTU transcription;
(c) introducing the RTU population into assay cells;
(d) isolating cellular RNA, and annealing same to an RTU detection oligonucleotide, which possesses a sequence complementary to the RTU reporter sequence and a facilitating adapter sequence for RNA sequencing by a nanopore sequencing device;
(e) optionally amplifying reporter RNA transcripts before the annealing (d) of said detection oligonucleotide via consecutive reverse transcription and PCR using RTU-specific primers;
(f) sequencing the annealed reporter RNA or its amplified cDNA through a nanopore sequencing device;
(g) analyzing sequencing output to identify and enumerate processing tags associated with individual RTUs; and
(h) calculating and generating an RTU activity profile as the frequency of the counted processing tags.

2. The method of claim 1, wherein said introducing (c) comprises transient or stable transfection, electroporation, or injection of the assay cells.

3. A method for real-time controlling of quality of multiplexed reporter construct detection in the method of claim 1, said real-time controlling method comprising:
(a) assessing RTU activity profiles calculated and generated in step (h) at two consecutive time points;
(b) calculating similarity of said RTU activity profiles using a Pearson correlation coefficient or Euclidean distance technique; and
(c) stopping the sequencing once the RTU profiles' similarity reaches a predetermined quality threshold.

4. A method for combined assessment of multiple reporter constructs and gene expression in living cells, said method comprising:
(a) use of a uniform population of reporter transcription units (RTU) and introduction of said RTU population into assay cells according to steps (a)-(c) defined in claim 1;
(b) isolating cellular mRNA;
(c) annealing one part of said isolated mRNA to an RTU detection oligonucleotide according to steps (d)-(e) defined in claim 1;
(d) annealing the other part of said isolated mRNA to an RNA detection oligonucleotide containing a poly(T) sequence complementary to the 3′ poly(A) RNA tail and an adapter sequence facilitating RNA sequencing by a nanopore sequencing device;
(e) mixing the annealed RNAs and sequencing the mix using a nanopore sequencing device, or
(f) alternatively, sequencing the annealed RNAs separately;
(g) analyzing the sequencing output to identify and enumerate processing tags associated with individual RTUs and calculating and generating an RTU activity profile as the frequency of the counted processing tags; and
(h) analyzing the sequencing output to calculate and generate an output of the expression of endogenous mRNA.

5. A kit for nanopore-based assessment of multiple reporter constructs, said kit comprising RTU constructs, detection reagents, and software for analyzing the sequencing output and calculating RTU activity profiles and gene expression according to the method of claim 1.
